(19) United States
(12) Patent Application Publication
(10) Pub. No.: US 2011/0288533 A1
(43) Pub. Date: Nov. 24, 2011

(54) Tip Controllable Guidewire Device

(75) Inventors: Durmus Koch, Englewood, NJ (US); Erol Eviner, Ringwood, NJ (US)

(73) Assignee: BIPORE MEDICAL DEVICES, INC., Northvale, NJ (US)

(21) Appl. No.: 12/783,706

(22) Filed: May 20, 2010

(51) Int. Cl. A61M 25/09

(52) U.S. Cl. 604/528

(57) ABSTRACT

A medical device for controlling the tip of an inner guidewire for advancement of an interventional device throughout the arterial system of a patient. The device includes an elongated housing with a closed rear end and an opposing open end. A traveller is disposed within the elongated housing and slides along the longitudinal axis of the elongated housing. A tubular shaft member that is attached to the proximal end of the traveller extends through the open end of the elongated housing. The proximal end of a guidewire is fixedly attached to the rear end of the elongated housing. The guidewire extends longitudinally through the elongated housing, through the traveller and is contained within the passageway of the tubular shaft member.

### FIELD OF THE INVENTION

The present invention relates to a device for controlling the tip of an inner guidewire for advancement of an interventional device throughout the arterial system of a patient. More particularly, the present invention relates to a device that includes an inner guidewire with a shaped distal tip that can be controlled by an operator to advance an interventional device through highly angulated pathways of the arterial system of a patient.

### BACKGROUND OF THE INVENTION

An introducer is used in the medical field to establish and provide access to the arterial system of a patient. Once established, the introducer provides a means of access to the arterial system for certain instruments or devices, and specifically, that device can be a guidewire that passes through the introducer wherein the distal end of the guidewire is located in the particular desired artery of the patient.

In a typical introduction of a guidewire into a patient, a needle is first inserted into the patient with its distal end positioned in the artery. A guidewire is then normally slid through the needle such that the distal end of the guidewire is located within the particular artery. At that point, the needle is removed while the guidewire remains in its position in the artery. An introducer, with a dilator, is then inserted over the guidewire into the artery. The dilator is then removed while the introducer is retained in that position with the guidewire passing therethrough such that other medical devices can be threaded over and along the guidewire through the introducer into the particular artery.

For example, a stent can be introduced through the introducer and located at the desired location with a balloon that is inflated to emplace the stent after which the balloon is removed.

The terms “proximal” and “distal” will be used herein to describe either opposing ends of a particular device, component, or element. The term “proximal” refers to an end of the device, component, or element that is closest to the operator. The term “distal” refers to an end of the device, component, or element that is farthest from the operator.

Previous guidewire insertion devices do not provide a distal tip end that can be controlled by an operator to provide easy advancement through highly angulated portions of the arterial system. For example, existing introducers and dilators with a pre-formed angulated distal tip end have been utilized with limited results and efficacy. Operators of these existing devices, however, have been unable to dynamically control the movement and curvature of the distal tip.

It is desired to provide a guidewire insertion device that has a distal tip end, where the shape and movement of the distal tip can be controlled and manipulated by the operator while inserted into a patient.

### SUMMARY OF THE INVENTION

The present invention is directed to a device for inserting a guidewire into the arterial system of a patient. The direction and angulation of the distal tip region of the guidewire can then be controlled by the operator of the device.

A tip-controllable guidewire device in accordance with the present invention generally includes an elongated housing with a closed rear end and an opposing open end. A traveller is disposed within the elongated housing and slides along the longitudinal axis of the elongated housing. A tubular shaft member that is attached to the proximal end of the traveller extends through the open end of the elongated housing. A guidewire extends longitudinally through the elongated housing, through the traveller and is contained within the passageway of the tubular shaft member. The proximal end of a guidewire is fixedly attached to the rear end of the elongated housing.

A mechanism attached to the elongated housing member used to move the traveller in a longitudinal direction, such as a threaded thumb wheel, allows for movement of the traveller within the elongated housing. Movement of the traveller causes the attached tubular shaft member to move longitudinally.

The threads on the thumb wheel engage with the grooves on the top surface of the traveller, such that rotating the thumb wheel in a distal direction causes the traveller to move in the proximal direction. In this case, tubular shaft member which is attached to the traveller is moved proximally and will expose the distal tip portion of the inner guidewire. Rotating the thumb wheel in an opposite, in this case proximal, direction causes the traveller to move in the distal direction. Here, tubular shaft member is moved distally on a longitudinal axis and will cause the distal tip portion of the inner guidewire to become enclosed by the tubular shaft member.

### BRIEF DESCRIPTION OF THE DRAWINGS

The present invention is illustrated by the accompanying drawings of various embodiments and the detailed description given below. The drawings should not be taken to limit the invention to the specific embodiments but are for explanation and clarity. The detailed description and drawings are merely illustrative of the invention, rather than limiting the scope of the invention being defined by the appended claims and equivalents thereof. The foregoing aspects and other attendant advantages of the present invention will become more readily appreciated by the detailed description taken in conjunction with the accompanying drawings.

FIG. 1 is a plan view, partially in section, of a guidewire device embodying features of the invention;

FIG. 2 is a transverse cross-sectional view taken along the lines of A-A shown FIG. 1.

### DETAILED DESCRIPTION OF THE INVENTION

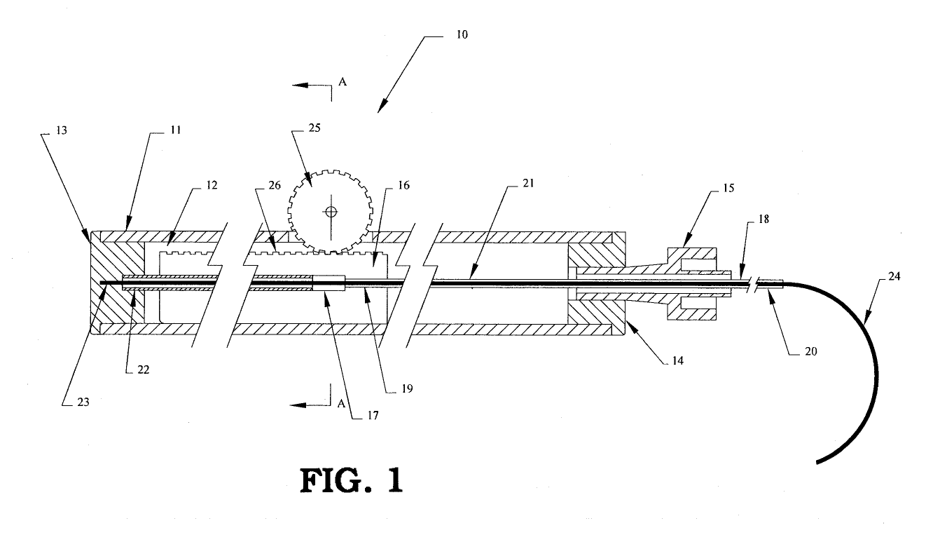

An apparatus 10 embodying features of the present invention is illustrated in FIG. 1 generally including an elongated housing 11 with inner cavity 12 extending between rear end 13 and opposing front end 14. Securely attached to the inner surface of front end 14 is Luer Lock extension 15. Traveller 16 is slidably disposed within inner cavity 12 and contains a hollow inner bore 17. Traveller 16 is a generally cylindrical-shaped, such as a piston. Both elongated housing 11 and traveller 16 are preferably made of a biocompatible thermoplastic material, such as high density polyethylene (HDPE), polypropylene, fluoropolymer, polyether block amide (PEBA), polyamide (PA), polyvinyl chloride (PVC), or a blend thereof.

Tubular shaft member 18 is attached to the proximal end of traveller 16 and extends proximally through inner cavity 12 and out of front end 14 of elongated housing 11. In a preferred embodiment, front end 14 includes a Luer Lock extension 15, through which tubular shaft member 18 extends. Tubular shaft member 18 has an elongate body with a proximal end 19, distal end 20, and an axial lumen 21, also used herein as “central passage.” Support tube 22 is fixedly attached to rear end 13. The inner bore 17 of traveller 16 is slidably disposed upon support tube 22.

Inner guidewire 23 is fixedly attached to rear end 13 and passes longitudinally through support tube 22 and into the inner bore 17 of traveller 16. Inner guidewire 23 continues through inner bore 17 of traveller 16 and into the proximal end of tubular shaft member 18. Inner guidewire 23 is contained within the axial lumen 21 of tubular shaft member 18 and extends to the distal end 20 of tubular shaft member 18. Inner guidewire 23 has a distal tip portion 24. In a preferred embodiment, inner guidewire 23 is a 0.021″ diameter stainless steel or nitinol type wire of appropriate length, but any size wire may be used.

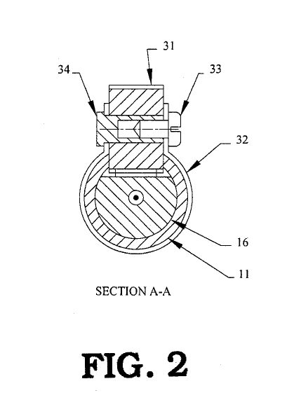

Traveller 16 moves within inner cavity 12, along the longitudinal axis of elongated housing 11. In a preferred embodiment, movement of traveller 16 in the longitudinal direction is accomplished by thumb wheel 25. In this embodiment, as seen in FIGS. 1 and 2, thumb wheel 25 has a threaded surface 31 and traveller 16 has a grooved upper surface 26. As thumb wheel 25 is rotated, the threaded surface 31 contacts with the grooved upper surface 26 of traveller 16 to cause longitudinal movement of traveller 16 within inner cavity 12.

With reference to FIGS. 1 and 2, thumb wheel strap 32 securely attaches thumb wheel 25 to elongate housing 11. Axial screws 33 and 34 attach thumb wheel 25 to thumb wheel strap 32.

As traveller 16 moves in a longitudinal direction, tubular member 18 slides over guidewire 23 to either expose or enclose the distal tip 24 of inner guidewire 23. Moving traveller 16 in the proximal direction causes tubular shaft member 18 to be moved in a proximal direction. Inner guidewire 23 remains stationary and thus as tubular shaft member 18 moves in the proximal direction, the distal tip portion 24 of the inner guidewire 23 will be exposed. When traveller 16 is moved in the distal direction, this causes tubular shaft member 18 to move distally over inner guidewire 23. When tubular shaft member 18 moves distally, the distal tip portion 24 of the inner guidewire 23 is enclosed by tubular shaft member 18.

In a typical operation of the present invention, an introducer sheath or dilator is placed over tubular member 18. The present invention, along with the introducer sheath or dilator is then inserted into the arterial system. To control the distal tip 24 of inner guidewire 13, the tubular member 18 is moved in a proximal direction to allow exposure the distal tip 24 of inner guidewire 23. This is accomplished by rotating thumb wheel 25 in a distal direction, thereby causing traveller 16 to slide in a proximal direction. As more of the distal tip 24 becomes exposed, the curvature of the distal tip 24 changes. The tip of either the introducer sheath or dilator can be manipulated by the change in curvature of distal tip 24 of inner guidewire 23. Thus, an operator can control the tip curvature for advancement of the inner guidewire through highly angulated and tortuous arterial system. The distal tip 24 of inner guidewire 23 may be shaped or configured in any manner suitable for advancement or retraction of the arterial system.

The invention has been described herein primarily directed to certain preferred embodiments, but those skilled in the art will recognize that variations and modifications may be made in form and detail without departing from the spirit and scope of the invention.

What is claimed is:

1. A device for controlling the tip of a guidewire for advancement through an arterial system, comprising:
a control handle having a hollow inner cavity;
an inner core wire fixedly attached to a proximal end of said control handle, wherein said inner core wire extends from within the hollow inner cavity of said control handle and out of a distal end of said control handle; and
a tubular member slidably disposed over said inner core wire, wherein said tubular member is operably attached to said control handle.

2. A device for controlling the tip of a guidewire for advancement through an arterial system, comprising:
an elongated housing having an inner cavity extending therein,
a traveller slidably disposed within the inner cavity of said housing, said traveller having an inner bore extending therein,
a tubular member fixedly attached on one end to said traveller,
a guidewire running through the inner cavity of said housing, into the inner bore of said traveller, and into said tubular member, and
a means to move said traveller within the inner cavity of said housing.

3. The device of claim 2 wherein said tubular member is fixedly attached on one end to a distal end of said traveller, the other end of said tubular member extending out of a distal end of said housing.

4. The device of claim 3 wherein said guidewire is secured to a proximal end of said housing.

5. The device of claim 3 wherein movement of said attached tubular member over said guidewire exposes a distal tip end of said guidewire.

6. The device of claim 3 wherein movement of said attached tubular member over said guidewire encloses a distal tip end of said guidewire.

7. The device of claim 3 wherein a top surface of said traveller comprises a plurality of grooves.

8. The device of claim 7 wherein said means to move said traveller within the inner cavity of said housing is a thumb wheel freely rotatable and securely attached to said elongated housing, said thumb wheel threadedly engaged to said top surface of said traveller through an exposed portion of said housing.

9. The device of claim 3 wherein said distal tip of said guidewire is shaped to a desired curvature.

10. The device of claim 3 wherein said means to move said traveller within the inner cavity of said housing is operably attached to said traveller and moves said traveller in a longitudinal direction thereby causing said attached tubular member to move over said guidewire.

11. The device of claim 10 wherein the amount of distal tip exposed is equal to the amount of longitudinal movement of said traveller.

12. The device of claim 2 wherein said means to move said traveller within the inner cavity of said housing further comprises a first position whereby a distal tip of said guidewire is extending through a distal end of said tubular member, and a second position whereby a distal tip of said guidewire is enclosed within a distal end of said tubular member.

13. The device of claim 3 wherein said elongated housing further comprises a closed end and an open end, wherein said open end includes a Luer lock attachment.

14. The device of claim 2 wherein said means to move said traveller within the inner cavity of said housing causes longitudinal movement of said attached tubular member in either a proximal direction or a distal direction to control the distal tip end of said guidewire.

15. The device of claim 9 wherein the longitudinal movement of said tubular member controls the amount of curvature of said distal tip end of said guidewire

16. The device of claim 2 further comprising a support tubular member having a proximal end and a distal end, the proximal end of said support tubular member being attached to the proximal end of said elongated housing.

17. The device of claim 16 wherein said traveller is slidably disposed upon said support tubular member.
